(19) United States
(12) Patent Application Publication
(10) Pub. No.: US 2011/0137180 A1
(43) Pub. Date: Jun. 9, 2011

(54) Systems and Methods for Fitting a Cochlear Implant System to a Patient Based on Stapedius Displacement

(75) Inventors: William L. Johnson, Santa Clarita, CA (US); Kinuko Masaki, Santa Monica, CA (US)

(73) Assignee: Advanced Bionics AG, c/o Froriep Renggli, Zug (CH)

(21) Appl. No.: 12/958,746

(22) Filed: Dec. 2, 2010

Related U.S. Application Data
(60) Provisional application No. 61/266,634, filed on Dec. 4, 2009.

(51) Int. Cl. A61B 5/05; A61B 6/00

(52) U.S. Cl. 600/478; 600/554

(57) ABSTRACT

An exemplary system includes an implantable cochlear stimulator configured to be implanted within a patient, a sound processor communicatively coupled to the implantable cochlear stimulator and configured to direct the implantable cochlear stimulator to generate and apply electrical stimulation to one or more stimulation sites within a cochlea of the patient, and a displacement sensor assembly configured to measure a displacement of the stapedius of the patient that occurs in response to the application of the electrical stimulation and transmit displacement data representative of the measured displacement to the sound processor. Corresponding systems and methods are also disclosed.

### RELATED APPLICATIONS

The present application claims priority under 35 U.S.C. §119(e) to U.S. Provisional Patent Application No. 61/266,634 by William L. Johnson, filed on Dec. 4, 2009, and entitled “Systems and Methods for Fitting a Cochlear Implant System to a Patient Based on Stapedius Tendon Strain,” the contents of which are hereby incorporated by reference in their entirety.

### BACKGROUND INFORMATION

The natural sense of hearing in human beings involves the use of hair cells in the cochlea that convert or transduce acoustic signals into auditory nerve impulses. Hearing loss, which may be due to many different causes, is generally of two types: conductive and sensorineural. Conductive hearing loss occurs when the normal mechanical pathways for sound to reach the hair cells in the cochlea are impeded. These sound pathways may be impeded, for example, by damage to the auditory ossicles. Conductive hearing loss may often be overcome through the use of conventional hearing aids that amplify sound so that acoustic signals can reach the hair cells within the cochlea. Some types of conductive hearing loss may also be treated by surgical procedures.

Sensorineural hearing loss, on the other hand, is caused by the absence or destruction of the hair cells in the cochlea which are needed to transduce acoustic signals into auditory nerve impulses. People who suffer from sensorineural hearing loss may be unable to derive significant benefit from conventional hearing aid systems, no matter how loud the acoustic stimulus. This is because the mechanism for transducing sound energy into auditory nerve impulses has been damaged. Thus, in the absence of properly functioning hair cells, auditory nerve impulses cannot be generated directly from sounds.

To overcome sensorineural hearing loss, numerous cochlear implant systems—or cochlear prostheses—have been developed. Cochlear implant systems bypass the hair cells in the cochlea by presenting electrical stimulation directly to the auditory nerve fibers. Direct stimulation of the auditory nerve fibers leads to the perception of sound in the brain and at least partial restoration of hearing function.

When a cochlear implant system is initially implanted in a patient, and during follow-up tests and checkups thereafter, it is usually necessary to fit the cochlear implant system to the patient. Such “fitting” includes adjustment of the base amplitude or intensity of the various stimuli generated by the cochlear implant system from the factory settings (or default values) to values that are most effective and comfortable for the patient. For example, the intensity or amplitude and/or duration of the individual stimulation pulses provided by the cochlear implant system may be mapped to an appropriate dynamic audio range so that the appropriate “loudness” of sensed audio signals is perceived. That is, loud sounds should be sensed by the patient at a level that is perceived as loud, but not painfully loud. Soft sounds should similarly be sensed by the patient at a level that is soft, but not so soft that the sounds are not perceived at all.

Fitting of a cochlear implant system to a patient is typically performed by an expert clinician who presents various stimuli to the patient and relies on subjective feedback from the patient as to how such stimuli are perceived. The subjective feedback typically takes the form of either verbal (adult) or non-verbal (child) feedback. Unfortunately, relying on subjective feedback in this manner is difficult, particularly for those patients who may have never heard sound before and/or who have never heard electrically-generated “sound”. For young children, the problem is exacerbated by short attention spans and an inability to understand or follow instructions. Moreover, many patients, such as infants and those with multiple disabilities, are completely unable to provide subjective feedback.

In addition, the optimal fitting parameters of a cochlear implant system may vary during a patient's lifetime. For example, in the developing nervous system of young children, frequent changes in the intensity of the stimuli may be required in order to optimize the cochlear implant system. The optimal fitting parameters may vary during a woman's menstrual cycle, or may vary with medication or illness. These changes may require frequent refitting sessions.

### SUMMARY

An exemplary system includes an implantable cochlear stimulator configured to be implanted within a patient, a sound processor communicatively coupled to the implantable cochlear stimulator and configured to direct the implantable cochlear stimulator to generate and apply electrical stimulation to one or more stimulation sites within a cochlea of the patient, and a displacement sensor assembly configured to measure a displacement of the stapedius of the patient that occurs in response to the application of the electrical stimulation and transmit displacement data representative of the measured displacement to the sound processor.

Another exemplary system includes an implantable cochlear stimulator configured to be implanted within a patient, a sound processor communicatively coupled to the implantable cochlear stimulator and configured to direct the implantable cochlear stimulator to generate and apply electrical stimulation to one or more stimulation sites within a cochlea of the patient, a fitting device communicatively coupled to the sound processor and configured to fit the implantable cochlear stimulator and the sound processor to the patient, and a strain gauge assembly selectively and communicatively coupled to the fitting device and configured to be coupled to a stapedius tendon of the patient, the strain gauge assembly further configured to measure a strain of the stapedius tendon that occurs in response to the application of the electrical stimulation and transmit strain data representative of the measured strain to the fitting device.

An exemplary method of fitting a cochlear prosthesis to a patient includes 1) applying, by an implantable cochlear stimulator, electrical stimulation to one or more stimulation sites within a cochlea of a patient, 2) measuring, by a displacement sensor assembly, a displacement of the stapedius of the patient that occurs in response to the applying of the electrical stimulation, 3) transmitting, by the displacement sensor assembly, displacement data representative of the measured displacement to an external control device configured to control an operation of the implantable cochlear stimulator, and 4) adjusting, by the external control device, one or more control parameters governing the operation of the implantable cochlear stimulator in accordance with the displacement data.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system according to principles described herein.

FIG. 2 illustrates exemplary components of a sound processor according to principles described herein.

FIG. 3 illustrates exemplary components of an implantable cochlear stimulator according to principles described herein.

FIG. 4 illustrates an exemplary implementation of the cochlear implant system of FIG. 1 according to principles described herein.

FIG. 5 shows the morphology of the stapedius muscle.

FIG. 6 shows an implanted displacement sensor assembly coupled to the stapedius tendon according to principles described herein.

FIG. 7A is a top view of an exemplary strain gauge assembly according to principles described herein.

FIG. 7B is a cross-sectional side view of the strain gauge assembly shown in FIG. 7A coupled to the stapedius tendon according to principles described herein.

FIG. 8A is a perspective view of another exemplary strain gauge assembly according to principles described herein.

FIG. 8B is a cross-sectional side view of the strain gauge assembly shown in FIG. 8A coupled to the stapedius tendon according to principles described herein.

FIG. 9 illustrates an exemplary configuration wherein a fitting device is selectively and communicatively coupled to a sound processor according to principles described herein.

FIG. 10 illustrates an exemplary method of fitting a cochlear prosthesis to a patient according to principles described herein.

### DETAILED DESCRIPTION

Systems and methods for fitting a cochlear implant system to a patient based on stapedius displacement are described herein. An exemplary system includes an implantable cochlear stimulator configured to be implanted within a patient, a sound processor communicatively coupled to the implantable cochlear stimulator and configured to direct the implantable cochlear stimulator to generate and apply electrical stimulation to one or more stimulation sites within a cochlea of the patient, and a displacement sensor assembly configured to be implanted within the patient (e.g., coupled to the stapedius tendon, the stapedius muscle, and/or the stapes of the patient). The displacement sensor assembly is further configured to measure a displacement (e.g., by measuring a movement, strain, tension, etc.) of the stapedius (e.g., the stapedius muscle and/or the stapedius tendon) that occurs in response to the application of the electrical stimulation and transmit displacement data representative of the measured displacement to the sound processor. The sound processor and/or a fitting device may utilize the displacement data to fit the cochlear implant system to the patient. For example, the sound processor and/or fitting device may utilize the displacement data to determine a range of intensities of electrical stimulation that are most comfortable for the patient and map audio signals presented to the patient to that range.

Numerous advantages are associated with the systems and methods described herein. For example, stapedius displacement serves as an objective and autonomous indicator of auditory stimulus volume perception by the patient and can be therefore utilized to objectively fit a cochlear implant system to a patient. Such fitting may be performed automatically in accordance with the systems and methods described herein, thus obviating the need for a patient to make repeated trips to a clinician's office in order to optimize the fitting of the patient's cochlear implant system.

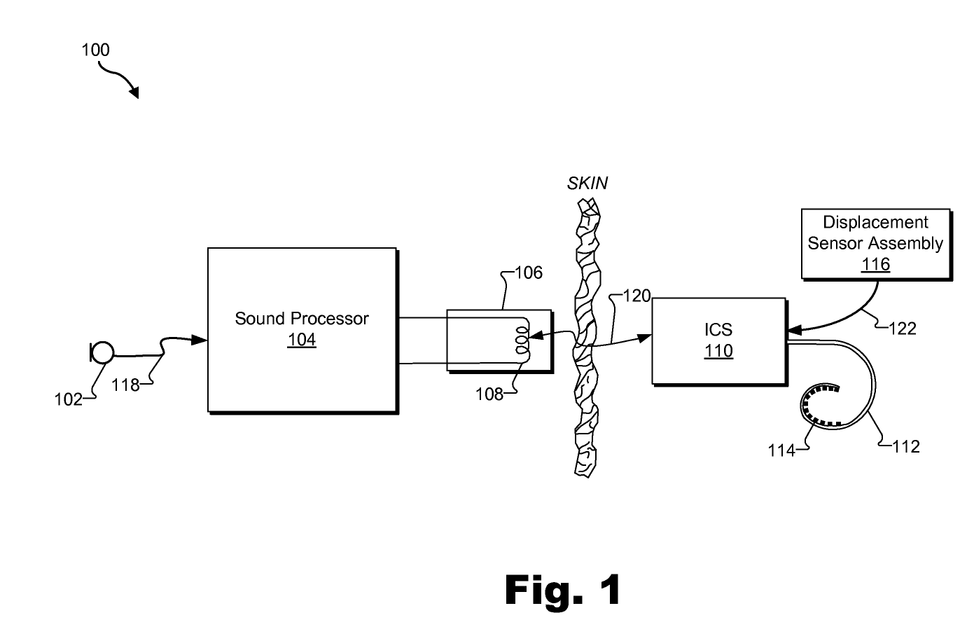

FIG. 1 illustrates an exemplary cochlear implant system 100. Cochlear implant system 100 may include a microphone 102, a sound processor 104, a headpiece 106 having a coil 108 disposed therein, an implantable cochlear stimulator (“ICS”) 110, a lead 112 with a plurality of electrodes 114 disposed thereon, and a displacement sensor assembly 116. Additional or alternative components may be included within cochlear implant system 100 as may serve a particular implementation.

As shown in FIG. 1, microphone 102, sound processor 104, and headpiece 106 may be located external to a cochlear implant patient. In some alternative examples, microphone 102 and/or sound processor 104 may be implanted within the patient. In such configurations, the need for headpiece 106 may be obviated.

Microphone 102 may detect an audio signal and convert the detected signal to a corresponding electrical signal. The electrical signal may be sent from microphone 102 to sound processor 104 via a communication link 118, which may include a telemetry link, a wire, and/or any other suitable communication link.

Sound processor 104 is configured to direct implantable cochlear stimulator 110 to generate and apply electrical stimulation (also referred to herein as “stimulation current”) to one or more stimulation sites within a cochlea of the patient. To this end, sound processor 104 may process the audio signal detected by microphone 102 in accordance with a selected sound processing strategy to generate appropriate stimulation parameters for controlling implantable cochlear stimulator 110. Sound processor 104 may include or be implemented within a behind-the-ear (“BTE”) unit, a portable speech processor (“PSP”), and/or any other sound processing unit as may serve a particular implementation. Exemplary components of sound processor 104 will be described in more detail below.

Sound processor 104 may be configured to transcutaneously transmit one or more control parameters and/or one or more power signals to implantable cochlear stimulator 110 with coil 108 by way of communication link 120. These control parameters may be configured to specify one or more stimulation parameters, operating parameters, and/or any other parameter as may serve a particular implementation. Exemplary control parameters include, but are not limited to, volume control parameters, program selection parameters, operational state parameters (e.g., parameters that turn a sound processor and/or an implantable cochlear stimulator on or off), audio input source selection parameters, fitting parameters, noise reduction parameters, microphone sensitivity parameters, microphone direction parameters, pitch parameters, timbre parameters, sound quality parameters, most comfortable current levels (“M levels”), threshold current levels, channel acoustic gain parameters, front and backend dynamic range parameters, current steering parameters, pulse rate values, pulse width values, frequency parameters, amplitude parameters, waveform parameters, electrode polarity parameters (i.e., anode-cathode assignment), location parameters (i.e., which electrode pair or electrode group receives the stimulation current), stimulation type parameters (i.e., monopolar, bipolar, or tripolar stimulation), burst pattern parameters (e.g., burst on time and burst off time), duty cycle parameters, spectral tilt parameters, filter parameters, and dynamic compression parameters. Sound processor 104 may also be configured to operate in accordance with one or more of the control parameters.

As shown in FIG. 1, coil 108 may be housed within headpiece 106, which may be affixed to a patient's head and positioned such that coil 108 is communicatively coupled to a corresponding coil included within implantable cochlear stimulator 110. In this manner, control parameters and power signals may be wirelessly transmitted between sound processor 104 and implantable cochlear stimulator 110 via communication link 120. It will be understood that data communication link 120 may include a bi-directional communication link and/or one or more dedicated uni-directional communication links. In some alternative embodiments, sound processor 104 and implantable cochlear stimulator 110 may be directly connected with one or more wires or the like.

Implantable cochlear stimulator 110 may be configured to generate electrical stimulation representative of an audio signal detected by microphone 102 in accordance with one or more stimulation parameters transmitted thereto by sound processing device 104. Implantable cochlear stimulator 110 may be further configured to apply the electrical stimulation to one or more stimulation sites within the cochlea via one or more electrodes 114 disposed along lead 112.

To facilitate application of the electrical stimulation generated by implantable cochlear stimulator 110, lead 112 may be inserted within a duct of the cochlea such that electrodes 114 are in communication with one or more stimulation sites within the cochlea. As used herein, the term “in communication with” refers to electrodes 114 being adjacent to, in the general vicinity of, in close proximity to, directly next to, or directly on the stimulation site. Any number of electrodes 114 (e.g., sixteen) may be disposed on lead 112 as may serve a particular implementation.

Displacement sensor assembly 116 may be configured to be implanted within a patient. For example, displacement sensor assembly 116 may be coupled to the stapedius tendon, the stapedius muscle, and/or the stapes (e.g., the stapes head) of the patient and measure a displacement of the stapedius (e.g., a displacement of the stapedius muscle and/or the stapedius tendon) that occurs in response to electrical stimulation being applied to the one or more stimulation sites by implantable cochlear stimulator 110. Displacement sensor assembly 116 may be further configured to transmit displacement data representative of the measured displacement to sound processor 104 by way of implantable cochlear stimulator 110. Hence, displacement sensor assembly 116 may be communicatively coupled to implantable cochlear stimulator 110 by way of communication link 122, which may include any wired or wireless link as may serve a particular implementation. As will be described in more detail below, sound processor 104 may fit and/or program implantable cochlear stimulator 110 in accordance with the transmitted displacement data. Additionally or alternatively, displacement sensor assembly 116 may transmit the displacement data directly to sound processor 104, without passing the data through implantable cochlear stimulator 110, and/or to an externally located fitting device configured to fit cochlear implant system 100 to the patient.

Displacement sensor assembly 116 may be implanted within a patient and configured to measure stapedius displacement in any suitable manner as may serve a particular implementation. Additionally or alternatively, displacement sensor assembly 116 may include an acute or non-implanted sensor. Exemplary implementations of displacement sensor assembly 116 will be described in more detail below.

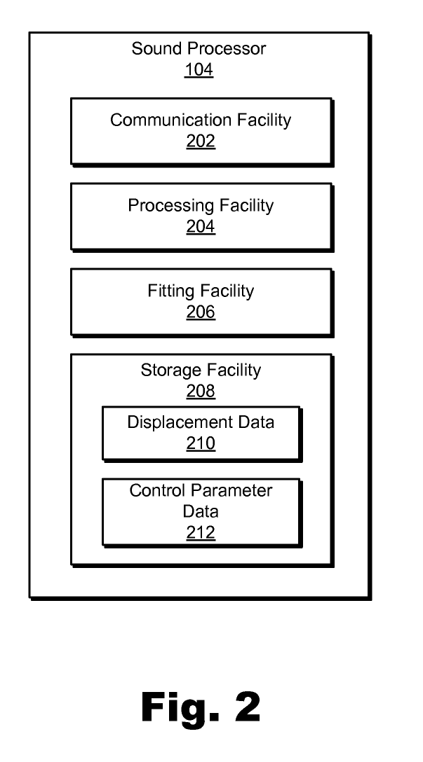

FIG. 2 illustrates exemplary components of sound processor 104. As shown in FIG. 2, sound processor 104 may include a communication facility 202, a processing facility 204, a fitting facility 206, and a storage facility 208, any or all of which may be in communication with one another using any suitable communication technologies. Each of these facilities 202-208 may include or be implemented by any combination of hardware, software, and/or firmware as may serve a particular implementation. For example, one or more of facilities 202-208 may include or be implemented by a computing device or processor configured to perform one or more of the functions described herein. It will also be recognized that one or more of facilities 202-208 may be optionally not included within sound processor 104. Facilities 202-208 will now be described in more detail.

Communication facility 202 may be configured to facilitate communication between sound processor 104 and implantable cochlear stimulator 110. For example, communication facility 202 may include transceiver components configured to wirelessly transmit data (e.g., control parameters and/or power signals) to implantable cochlear stimulator 110 and/or wirelessly receive data (e.g., displacement data and/or status data) from implantable cochlear stimulator 110 by way of coil 108. Communication facility 202 may be further configured to facilitate communication between sound processor 104 and displacement sensor assembly 116. For example, communication facility 202 may be configured to receive displacement data directly from displacement sensor assembly 116 by way of a wired or wireless communication link and/or by way of implantable cochlear stimulator 110.

Processing facility 204 may be configured to perform one or more signal processing heuristics on an audio signal presented to the patient. For example, processing facility 204 may perform one or more pre-processing operations, spectral analysis operations, noise reduction operations, mapping operations, and/or any other types of signal processing operations on a detected audio signal as may serve a particular implementation. In some examples, processing facility 204 may generate one or more control parameters governing an operation of implantable cochlear stimulator 110 (e.g., one or more stimulation parameters defining the electrical stimulation to be generated and applied by implantable cochlear stimulator 110).

Fitting facility 206 may be configured to fit implantable cochlear stimulator 110 to a patient in accordance with displacement data detected by displacement sensor assembly 116. As used herein, “fitting” implantable cochlear stimulator 110 to a patient refers to adjusting (i.e., optimizing) one or more control parameters governing an operation of implantable cochlear stimulator 110 or otherwise programming implantable cochlear stimulator 110 to meet the specific needs of the patient. For example, fitting facility 206 may be configured to utilize the displacement data to determine a range of stimulus intensities that are most comfortable to the patient.

The fitting may be performed by fitting facility 206 at any suitable time as may serve a particular implementation. For example, fitting facility 206 may fit implantable cochlear stimulator 110 to a patient during an initial fitting procedure that occurs soon after implantable cochlear stimulator 110 is implanted within the patient and/or at any subsequent time as may serve a particular implementation.

In some examples, fitting facility 206 may be configured to automatically perform the fitting procedure in accordance with the displacement data without input provided by a clinician or other user. Additionally or alternatively, fitting facility 206 may be configured to interface with a fitting device utilized by a clinician to fit cochlear implant system 100 to the patient.

Storage facility 208 may be configured to maintain displacement data 210 representative of the fitting data detected by displacement sensor assembly 116 and control parameter data 212 representative of one or more control parameters generated by processing facility 204. Storage facility 208 may be configured to maintain additional or alternative data as may serve a particular implementation.

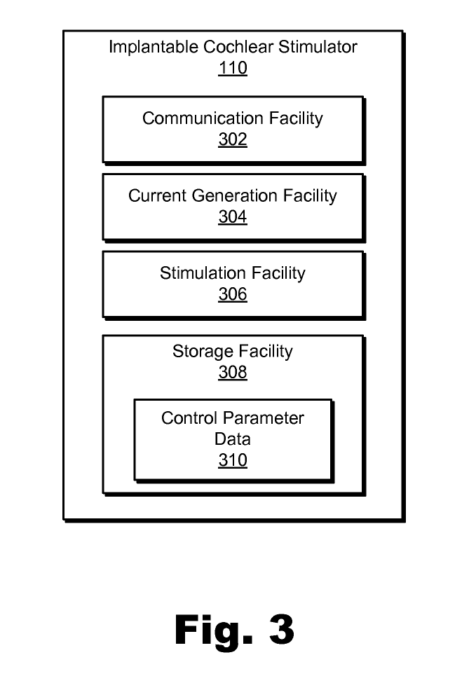

FIG. 3 illustrates exemplary components of implantable cochlear stimulator 110. As shown in FIG. 3, implantable cochlear stimulator 110 may include a communication facility 302, a current generation facility 304, a stimulation facility 306, and a storage facility 308, which may be in communication with one another using any suitable communication technologies. Each of these facilities 302-308 may include any combination of hardware, software, and/or firmware as may serve a particular implementation. For example, one or more of facilities 302-308 may include a computing device or processor configured to perform one or more of the functions described herein. Facilities 302-308 will now be described in more detail.

Communication facility 302 may be configured to facilitate communication between implantable cochlear stimulator 110 and sound processor 104. For example, communication facility 302 may include one or more coils configured to receive control signals and/or power via one or more communication links to implantable cochlear stimulator 110. Communication facility 302 may additionally or alternatively be configured to transmit one or more status signals and/or other data to sound processor 104.

Communication facility 302 may be further configured to facilitate communication between implantable cochlear stimulator 110 and displacement sensor assembly 116. For example, communication facility 302 may receive displacement data detected by displacement sensor assembly 116 and then transmit the displacement data to sound processor 104.

Current generation facility 304 may be configured to generate stimulation current in accordance with one or more stimulation parameters received from sound processor 104. To this end, current generation facility 304 may include one or more current generators and/or any other circuitry configured to generate stimulation current. For example, current generation facility 304 may include an array of independent current generators each corresponding to a distinct electrode 114 or channel.

Stimulation facility 306 may be configured to facilitate application of the stimulation current generated by current generation facility 304 to one or more stimulation sites within the patient in accordance with one or more stimulation parameters received from sound processor 104. To this end, stimulation facility 306 may be configured to interface with one or more electrodes (e.g., disposed on a lead that may be inserted within the patient (e.g., within the cochlea).

Storage facility 308 may be configured to maintain control parameter data 310 as received from sound processor 104. Control parameter data 310 may be representative of one or more control parameters configured to govern one or more operations of implantable cochlear stimulator 110. For example, control parameter data 310 may include data representative of one or more stimulation parameters configured to define the electrical stimulation generated and applied by implantable cochlear stimulator 110. Storage facility 308 may be configured to maintain additional or alternative data as may serve a particular implementation.

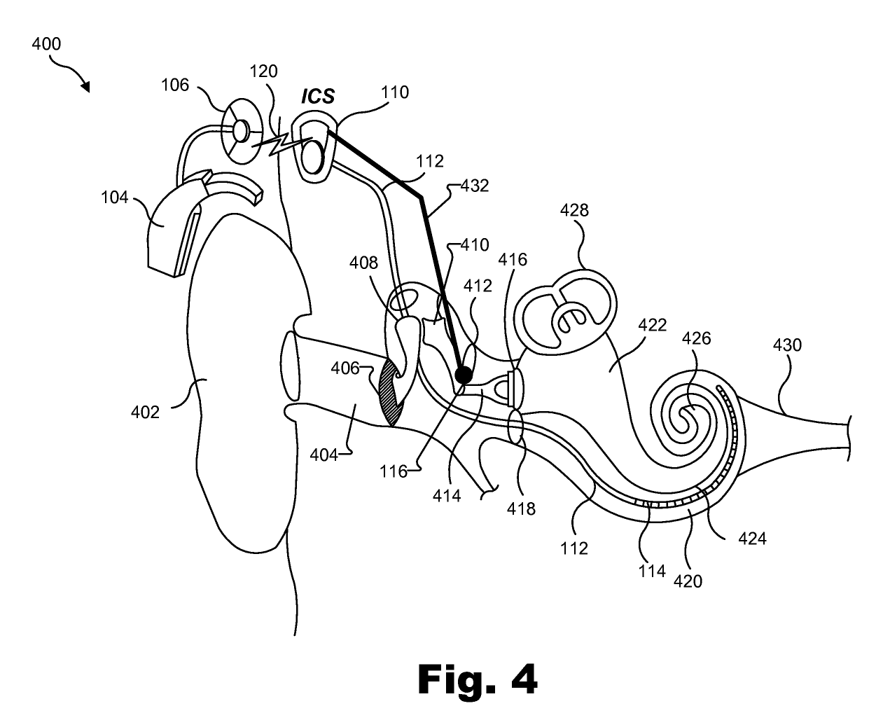

FIG. 4 illustrates an exemplary implementation 400 of cochlear implant system 100 wherein implantable cochlear stimulator 110 and displacement sensor assembly 116 are implanted within a patient. As shown in FIG. 4, sound processor 104 may be located external to the patient and mounted behind the ear. Headpiece 106 is positioned such that a coil (e.g., coil 108 in FIG. 1) disposed therein may be inductively coupled to a corresponding coil included within implantable cochlear stimulator 110. In this manner, sound processor 104 may transmit control parameters to implantable cochlear stimulator 110 by way of communication link 120. Lead 112 is also implanted within the patient such that electrodes 114 are disposed within the cochlea. Implantable cochlear stimulator 110 may generate and apply electrical stimulation to one or more stimulation sites within the cochlea via electrodes 114.

FIG. 4 also shows various anatomical features associated with the perception of sound, some of which will now be described in order to facilitate an understanding of the systems and methods described herein.

As shown in FIG. 4, the ear may include a pinna 402, ear canal 404, tympanic membrane 406, malleus 408, incus 410, stapedius muscle 412, stapes 414, oval window 416, round window 418, various structures within the cochlea (e.g., the scala tympani 420, the scala vestibuli 422, the basilar membrane 424, and the helicotrema 426), the labyrinth 428, and the auditory nerve 430. In a normally functioning ear, the tympanic membrane 406 vibrates in response to ambient sound, and via the ossicular chain (which includes the malleus 408, the incus 410, and the stapes 414), the vibration is transferred to the oval window 416.

The stapedius muscle 412 acts as a hearing damper by exerting a force on the stapes 414 and thereby increasing the impedance of the middle ear when uncomfortable sound levels are detected. Its activity level is therefore an autonomous indicator of auditory stimulus volume perception by the patient and can be utilized to fit cochlear implant system 100 to a patient.

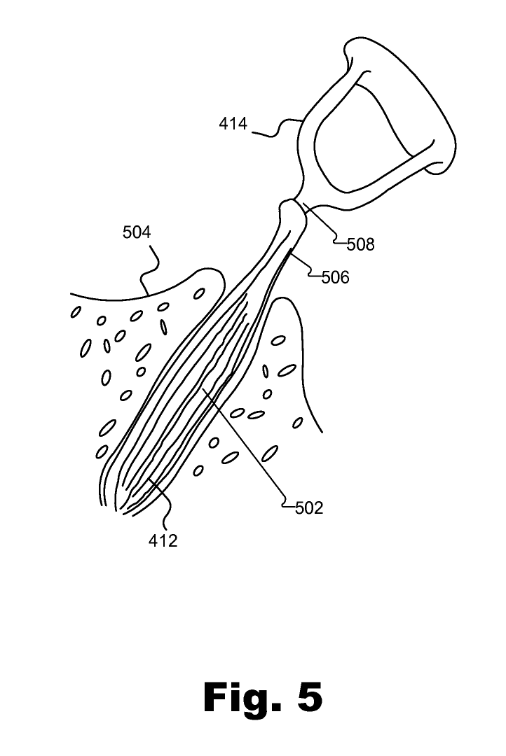

FIG. 5 shows the morphology of the stapedius muscle 412 and shows that a belly 502 of the stapedius muscle 412 is surrounded by a bone structure referred to as the pyramidal eminence 504. The belly 502 of the stapedius muscle 412 is connected to the stapes 414 by the stapedius tendon 506, a portion of which is not surrounded by the pyramidal eminence 504. The term “stapedius,” as used herein, refers to the stapedius muscle 412 and/or the stapedius tendon 506. The stapedius tendon 506 is attached to the stapes 414. In particular, the stapedius tendon 506 is attached to a head 508 of the stapes 414.

The stapedius tendon 506 is elastic in nature. Hence, activity (e.g., contraction or relaxation) of the stapedius muscle 412 may cause corresponding activity in the stapedius tendon 506 (e.g., stretching or contracting). The resulting displacement of the stapedius muscle 412 and/or the stapedius tendon 506 may be measured and used to determine a corresponding amount of stapedius muscle activity that occurs in response to an application of electrical stimulation to one or more sites within the cochlea.

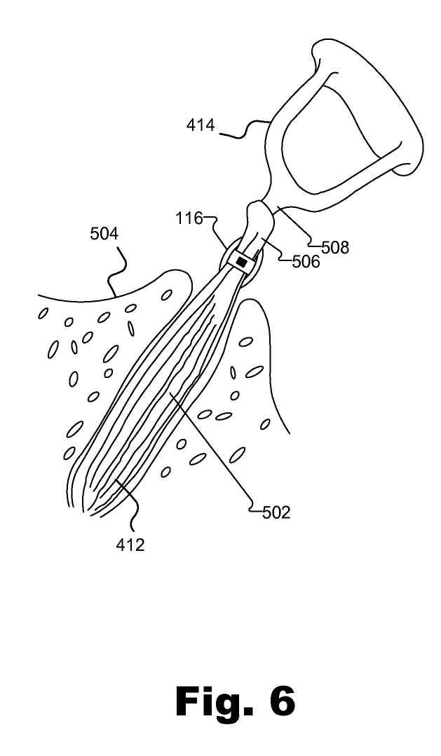

To measure stapedius displacement (i.e., displacement of the stapedius muscle 412 and/or the stapedius tendon 506), displacement sensor assembly 116 may be surgically implanted within the patient. In some examples, displacement sensor assembly 116 is coupled (i.e., attached) to the stapedius tendon 506. To illustrate, FIG. 6 shows an implanted displacement sensor assembly 116 coupled to the stapedius tendon 506. Additionally or alternatively, displacement sensor assembly 116 may be coupled to the stapedius muscle 412, the stapes head 508, and/or any other portion of the stapes 414.

Returning to FIG. 4, this figure also shows the general location of an implanted displacement sensor assembly 116, although the stapedius tendon 506 is not specifically illustrated in FIG. 4. Displacement sensor assembly 116 may be communicatively coupled to implantable cochlear stimulator 110 with wire 432. In this manner, displacement sensor assembly 116 may transmit detected displacement data to implantable cochlear stimulator 110, which may in turn wirelessly transmit the displacement data to sound processor 104. Additionally or alternatively, displacement sensor assembly 116 may be configured to communicate directly with sound processor 104 and/or any other device (e.g., a fitting device) by way of a wired or wireless communication link. As described previously, sound processor 104 may utilize the displacement data to fit implantable cochlear stimulator 110 to the patient (e.g., adjust one or more control parameter such that audio signals presented are mapped to a range of stimulus intensities that are most comfortable for the patient).

Various examples of displacement sensor assembly 116 will now be described. It will be recognized that the exemplary displacement sensor assemblies 116 described herein are merely illustrative of the many different displacement sensor assemblies that may be used in connection with the systems and methods described herein.

In some examples, displacement sensor assembly 116 may include a strain gauge assembly configured to detect a strain of the stapedius (in particular, the stapedius tendon). It will be recognized that displacement and strain are related. For example, displacement may be represented by Δx (i.e., delta x), while strain may be represented by (Δx)/X, where Δx represents a change in length (caused by stretching or contracting of the stapedius tendon and X represents a reference length (e.g., a total length) of the stapedius tendon. Hence, stapedius displacement may be detected by detecting stapedius tendon strain.

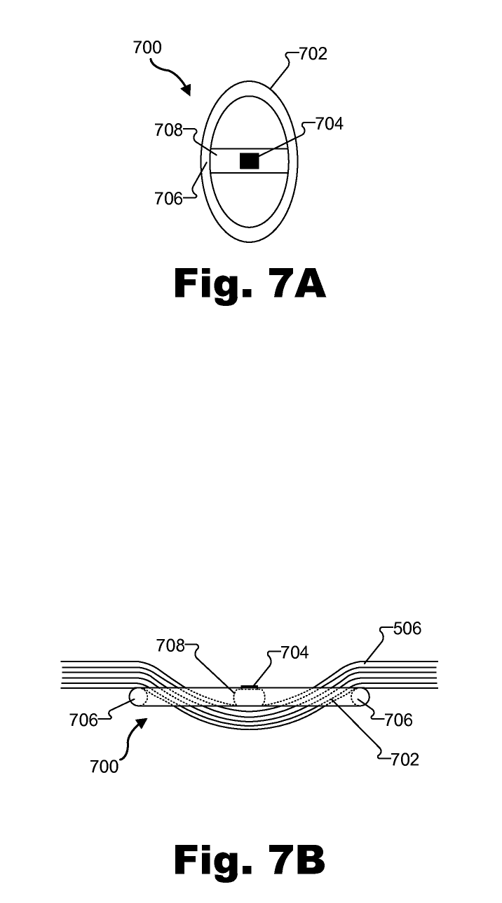

FIG. 7A is a top view of an exemplary strain gauge assembly 700 that includes a buckle frame 702 and a strain gauge 704 coupled to the buckle frame 702. As shown in FIG. 7A, buckle frame 702 may include an outer frame 706 and at least one removable crossbar 708 seated upon outer frame 706. Outer frame 706 may be oval in shape, as shown in FIG. 7A. Alternatively, outer frame 706 may have any other suitable shape (e.g., rectangular). Outer frame 706 and crossbar 708 may be made out of any suitable material sufficiently flexible to deform in response to stapedius tendon strain.

FIG. 7B is a cross-sectional side view of the strain gauge assembly 700 shown in FIG. 7A coupled to the stapedius tendon 506. As shown in FIG. 7B, buckle frame 702 has been coupled to the stapedius tendon 506 such that the stapedius tendon 506 passes over outer frame 706 and under crossbar 708. Crossbar 708 may be secured to outer frame 706 to lock buckle frame 702 in position.

With buckle frame 702 coupled to stapedius tendon 506 in this manner, a change in tension of stapedius tendon 506 causes buckle frame 702 to deform. The deformation of buckle frame 702 is detected by strain gauge 704, which may be configured to produce an output signal (e.g., a voltage) proportional to the tension developed within buckle frame 702. This output signal may be transmitted by strain gauge 704 to implantable cochlear stimulator 110, sound processor 104, and/or a fitting device as strain data, which may be used in a similar manner as displacement data. To this end, strain gauge 704 may be electrically coupled to one or more of these devices by way of a wire or the like.

It will be recognized that other buckle-type strain gauge assemblies 700 may be used in connection with the systems and methods described herein. For example, an alternative buckle-type strain gauge assembly 700 may include an E-shaped buckle frame. An E-shaped buckle frame is shaped like a capital “E” and thus does not need a removable crossbar in order to be coupled to the stapedius tendon.

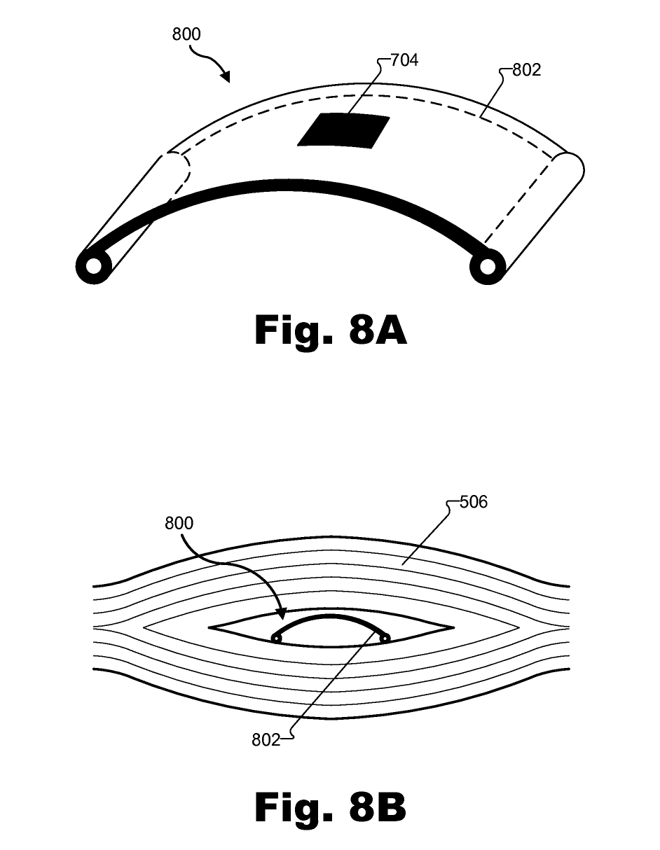

FIG. 8A is a perspective view of another exemplary strain gauge assembly 800 that may be used in connection with the systems and methods described herein. As shown in FIG. 8A, the strain gauge assembly 800 may include a pre-curved member 802 configured to be implanted at least partially within the stapedius tendon and bend in response to the strain of the stapedius tendon. Pre-curved member 802 may be shaped in order to realize three-point bending in response to downward force exerted on top of pre-curved member 802 and upward force exerted from beneath both ends of pre-curved member 802. Pre-curved member 802 may be alternatively shaped (i.e., in the form of a cylinder with a slit extending along a length thereof) in order to realize two-point bending.

Strain gauge 704 may be coupled to pre-curved member 802 and configured to measure stapedius tendon strain by detecting the bend of pre-curved member 802 that occurs in response to the strain of the stapedius tendon. Strain gauge 704 may be configured to generate strain data (or displacement data) based on the detected bend and transmit the strain data to implantable cochlear stimulator 110, sound processor 104, and/or a fitting device. To this end, strain gauge 704 may be electrically coupled to one or more of these devices by way of a wire or the like.

FIG. 8B is a cross-sectional side view of the strain gauge assembly 800 shown in FIG. 8A coupled to the stapedius tendon 506. As shown in FIG. 8B, pre-curved member 802 has been implanted entirely within the stapedius tendon 506. In this manner, tension within stapedius tendon 506 causes pre-curved member 802 to bend.

Other displacement sensor assemblies 116 may alternatively be used. For example, displacement sensor assembly 116 may include a fiber optic gauge assembly. The fiber optic gauge assembly may include an optical fiber configured to be inserted entirely through a cross-section of the stapedius tendon or muscle and bend or compress in response to stapedius displacement or strain. Additionally or alternatively, the optical fiber may be configured to be wrapped at least partially around the stapedius tendon or muscle. The fiber optic gauge assembly may further include a transmitter-receiver unit and a detector unit. The transmitter-receiver unit may be attached to both ends of the optical fiber and may be configured to pass a light signal through a core of the optical fiber. The detector unit may be configured to measure the stapedius displacement by detecting a modulation of an intensity (or any other property such as wavelength or phase) of the light signal that occurs in response to the bending or compressing of the optical fiber. This modulation is proportional to the amount of stapedius displacement or strain. The transmitter-receiver unit and/or the detection unit may be included or otherwise implemented by implantable cochlear stimulator 110, sound processor 104, and/or any other device as may serve a particular implementation.

Additionally or alternatively, displacement sensor assembly 116 may include an inductive gauge assembly (also referred to as an inductive sensor or transducer). The inductive gauge assembly may include a coil, which may surround the stapedius tendon or be positioned at any other suitable location. In some examples, ferromagnetic material may be disposed within the coil. The inductive gauge assembly may further include a sensor, which may be placed on or near the stapedius tendon, and which may be configured to measure a change of flux through the coil caused by either deformation of the coil or a change in the material inside the coil. The change in flux is proportional to stapedius tendon displacement and may therefore be used to derive displacement data that may be transmitted to sound processor 104.

Additionally or alternatively, displacement sensor assembly 116 may include a capacitive gauge assembly. The capacitive gauge assembly may include at least two conductive members placed on opposite sides of the stapedius tendon. The capacitive gauge assembly may further include a sensor configured to measure changes in capacitance due to a displacement of the capacitive members that occurs as a result of stapedius tendon strain or contraction. The measured changes may be used to derive displacement data that may be transmitted to sound processor 104.

Additionally or alternatively, displacement sensor assembly 116 may include a piezo-electric sensor assembly. The piezo-electric sensor assembly may be configured to produce and record voltage when subject to stapedius displacement or strain. The recorded voltage may be used to derive displacement data that may be transmitted to sound processor 104.

Additionally or alternatively, displacement sensor assembly 116 may include an accelerometer sensor assembly configured to measure an acceleration of the stapedius tendon or muscle. Acceleration and displacement are related by double integration. Hence, displacement data may be derived from the measured acceleration.

Displacement sensor assembly 116 may additionally or alternatively include any other type of sensor such as, but not limited to, a linear variable differential transformer (“LVDT”), a differential variable reluctance transformer (“DVRT”), and/or any other component configured to measure displacement by measuring variation of electrical resistance, magnetic field, or light flow.

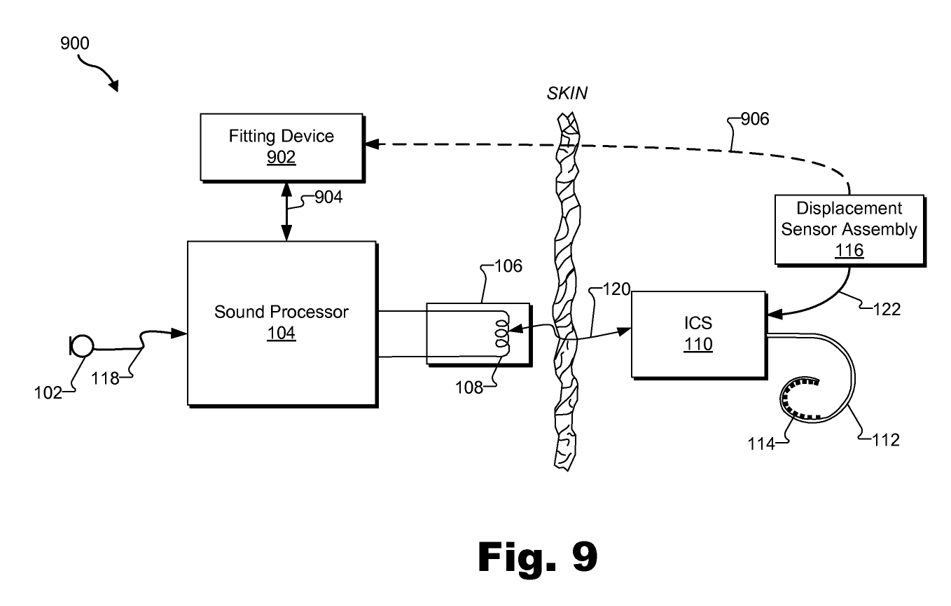

FIG. 9 illustrates an exemplary configuration wherein a fitting device 902 is selectively and communicatively coupled to sound processor 104 by way of communication link 904. Fitting device 902 may include any computing device configured to facilitate fitting of cochlear implant system 100 to a patient. For example, fitting device 902 may include a personal computer, a handheld device, a mobile device (e.g., a mobile phone), or any other suitable computing device. In some examples, fitting device 902 may be used by a clinician or other person trained to perform one or more fitting procedures.

In some examples, fitting device 902 may be configured to fit cochlear implant system 100 (i.e., sound processor 104 and/or implantable cochlear stimulator 110) to a patient in accordance with displacement data detected by displacement sensor assembly 116. To this end, displacement sensor assembly 116 may be configured to transmit displacement data directly to fitting device 902 by way of communication link 906. Communication link 906 may include a wireless and/or wired communication link as may serve a particular implementation. Displacement sensor assembly 116 may additionally or alternatively transmit displacement data to sound processor 104 by way of implantable cochlear stimulator 110. Sound processor 104 may then transmit the displacement data to fitting device 902 by way of communication link 904.

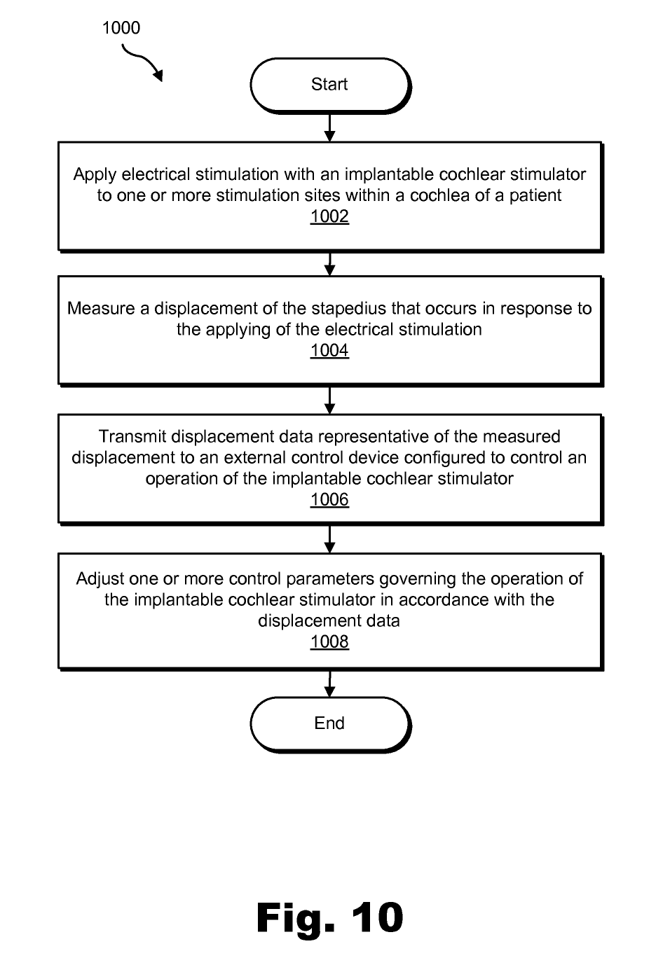

FIG. 10 illustrates an exemplary method 1000 of fitting a cochlear prosthesis to a patient. While FIG. 10 illustrates exemplary steps according to one embodiment, other embodiments may omit, add to, reorder, and/or modify any of the steps shown in FIG. 10.

In step 1002, electrical stimulation is applied with an implantable cochlear stimulator to one or more stimulation sites within a cochlea of a patient. For example, sound processor 104 may direct implantable cochlear stimulator 110 to apply electrical stimulation to one or more stimulation sites via one or more of electrodes 114.

In step 1004, a displacement of the stapedius that occurs in response to the applying of the electrical stimulation is measured. For example, displacement sensor assembly 116 may be placed on or near the stapedius tendon, stapedius muscle, and/or stapes and configured to measure the stapedius displacement in any of the ways described herein.

In step 1006, displacement data representative of the measured displacement is transmitted to an external control device configured to control an operation of the implantable cochlear stimulator. The external control device may include sound processor 104, fitting device 902, and/or any other device as may serve a particular implementation. In some examples, the displacement data may be transmitted to the external device by way of the implantable cochlear stimulator.

In step 1008, one or more control parameters governing the operation of the implantable cochlear stimulator are adjusted in accordance with the displacement data. The one or more control parameters may be adjusted in any of the ways described herein. For example, the external device may utilize the displacement data to determine a range of stimulus intensities that are most comfortable to the patient and then map audio signals received by sound processor 104 to that range. In some examples, the adjusting of the one or more control parameters is configured to fit the cochlear prosthesis (i.e., sound processor 104 and/or implantable cochlear stimulator 110) to the patient.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
an implantable cochlear stimulator configured to be implanted within a patient;
a sound processor communicatively coupled to the implantable cochlear stimulator and configured to direct the implantable cochlear stimulator to generate and apply electrical stimulation to one or more stimulation sites within a cochlea of the patient; and
a displacement sensor assembly configured to measure a displacement of the stapedius of the patient that occurs in response to the application of the electrical stimulation and transmit displacement data representative of the measured displacement to the sound processor.

2. The system of claim 1, wherein the displacement sensor assembly is configured to be coupled to at least one of a stapedius tendon, a stapedius muscle, and a stapes of the patient.

3. The system of claim 1, wherein the sound processor is further configured to automatically fit the implantable cochlear stimulator to the patient in accordance with the displacement data.

4. The system of claim 1, wherein the sound processor is further configured to adjust one or more control parameters governing an operation of at least one of the sound processor and the implantable cochlear stimulator in accordance with the displacement data.

5. The system of claim 4, wherein the one or more control parameters comprise a most comfortable current level.

6. The system of claim 1, wherein the sound processor is further configured to determine a range of intensities of the electrical stimulation that are most comfortable for the patient in accordance with the displacement data.

7. The system of claim 1, further comprising a fitting device communicatively coupled to the sound processor, wherein the sound processor is configured to transmit the displacement data to the fitting device, and wherein the fitting device is configured to fit the implantable cochlear stimulator and the sound processor to the patient in accordance with the displacement data.

8. The system of claim 1, wherein the displacement sensor assembly is electrically coupled to the implantable cochlear stimulator and configured to transmit the displacement data to the sound processor by way of the implantable cochlear stimulator.

9. The system of claim 1, wherein the displacement sensor assembly is configured to wirelessly transmit the displacement data to the sound processor.

10. The system of claim 1, wherein the displacement sensor assembly is configured to measure the displacement of the stapedius by measuring a strain of a stapedius tendon of the patient.

11. The system of claim 10, wherein the displacement sensor assembly comprises a strain gauge assembly comprising:
a buckle frame configured to be coupled to an outer portion of the stapedius tendon and deform in response to the displacement of the stapedius tendon; and
a strain gauge coupled to the buckle frame and configured to measure the strain by detecting the deformation of the buckle frame.

12. The system of claim 10, wherein the displacement sensor assembly comprises a strain gauge assembly comprising:
a pre-curved member configured to be implanted at least partially within the stapedius tendon and bend in response to the strain of the stapedius tendon; and
a strain gauge coupled to the pre-curved member and configured to measure the strain by detecting the bend of the pre-curved member.

13. The system of claim 1, wherein the displacement sensor assembly comprises a fiber optic gauge assembly that comprises:
an optical fiber configured to be inserted entirely through a cross-section of the stapedius tendon or wrapped at least partially around the stapedius tendon and bend or compress in response to the displacement of the stapedius tendon;
a transmitter-receiver unit attached to both ends of the optical fiber and configured to pass a light signal through a core of the optical fiber; and
a detector unit configured to measure the displacement by detecting a modulation of a property of the light signal that occurs in response to the bending or compressing of the optical fiber.

14. The system of claim 1, wherein the displacement sensor assembly comprises at least one of an inductive gauge assembly, a capacitive gauge assembly, a piezo-electric sensor assembly, and an accelerometer sensor assembly.

15. A system comprising:
an implantable cochlear stimulator configured to be implanted within a patient;
a sound processor communicatively coupled to the implantable cochlear stimulator and configured to direct the implantable cochlear stimulator to generate and apply electrical stimulation to one or more stimulation sites within a cochlea of the patient;
a fitting device communicatively coupled to the sound processor and configured to fit the implantable cochlear stimulator and the sound processor to the patient; and
a strain gauge assembly selectively and communicatively coupled to the fitting device and configured to be coupled to a stapedius tendon of the patient, the strain gauge assembly further configured to measure a strain of the stapedius tendon that occurs in response to the application of the electrical stimulation and transmit strain data representative of the measured strain to the fitting device.

16. The system of claim 15, wherein the fitting device is configured to fit the implantable cochlear stimulator and the sound processor to the patient in accordance with the strain data.

17. The system of claim 15, wherein the strain gauge assembly comprises:
a buckle frame configured to be coupled to an outer portion of the stapedius tendon and deform in response to the strain of the stapedius tendon; and
a strain gauge coupled to the buckle frame and configured to measure the strain by detecting the deformation of the buckle frame.

18. The system of claim 15, wherein the strain gauge assembly comprises:
a pre-curved member configured to be implanted at least partially within the stapedius tendon and bend in response to the strain of the stapedius tendon; and
a strain gauge coupled to the pre-curved member and configured to measure the strain by detecting the bend of the pre-curved member.

19. A method of fitting a cochlear prosthesis to a patient, the method comprising:
applying, by an implantable cochlear stimulator, electrical stimulation to one or more stimulation sites within a cochlea of a patient;
measuring, by a displacement sensor assembly, a displacement of the stapedius of the patient that occurs in response to the applying of the electrical stimulation;
transmitting, by the displacement sensor assembly, displacement data representative of the measured displacement to an external control device configured to control an operation of the implantable cochlear stimulator; and
adjusting, by the external control device, one or more control parameters governing the operation of the implantable cochlear stimulator in accordance with the displacement data.

20. The method of claim 19, further comprising determining, by the external control device, a range of intensities of the electrical stimulation that are most comfortable for the patient in accordance with the displacement data.
